(19) United States
(12) Patent Application Publication
(10) Pub. No.: US 2026/0083595 A1
(43) Pub. Date: Mar. 26, 2026

(54) Haptic Gloves for Enhanced Music Experience and Medical Applications

(71) Applicant: Karmel Graham, Beverly Hills, CA (US)

(72) Inventor: Karmel Graham, Beverly Hills, CA (US)

(21) Appl. No.: 18/895,413

(22) Filed: Sep. 25, 2024

(51) Int. Cl. A61F 11/04; A61H 23/00

(52) U.S. Cl. CPC A61F 11/045; A61H 23/006; A61H 2201/165; A61H 2201/5007

(57) ABSTRACT

This invention relates to a set of haptic gloves embedded with a taptic engine that connects to an iPhone via Bluetooth. The gloves are designed to vibrate in synchronization with the haptic music feature on iPhones, providing an enhanced sensory experience for users, particularly benefiting those who are deaf or hard of hearing. Additionally, the invention has potential applications in the medical field, specifically for rehabilitation purposes.

### BACKGROUND OF THE INVENTION

Music appreciation has traditionally relied heavily on auditory perception. However, this limits the experience for individuals with hearing impairments. Existing solutions for tactile music experiences are often limited in their functionality or lack integration with popular music platforms. Furthermore, haptic feedback devices in medical rehabilitation often lack the precision and customization options needed for effective treatment.

### SUMMARY OF INVENTION

The present invention addresses these challenges by providing a wearable haptic system that:
1. Seamlessly integrates with Apple's existing haptic music feature.
2. Offers a more immersive music experience through tactile sensations.
3. Provides customizable vibration patterns for various applications.
4. Has potential uses in medical rehabilitation.

### DETAILED DESCRIPTION OF THE INVENTION

The haptic gloves consist of the following key components:
Taptic Engine: Embedded within the gloves, capable of producing precise vibrations.
Microcontroller with Bluetooth Module: Enables wireless connection to an iPhone. Something like an Arduino Nano 33 BLE or an ESP32.
Control Unit, Wires, Connectors: Processes signals from the iPhone and controls the taptic engine. Connects the taptic engine to the microcontroller.
Power Source: Rechargeable battery for portability that powers the microcontroller and taptic engine.
Glove: A comfortable glove, preferably using a lightweight smart fabric, that can house the components.

The gloves connect to an iPhone via Bluetooth and synchronize with the device's haptic music feature. When a user plays music through Apple Music with the haptic feature enabled, the gloves vibrate in identical patterns to those produced by the iPhone.

### Functionality

1. Music Mode: Vibrates in sync with music, translating auditory rhythms into tactile sensations.
2. Customization: Allows users to adjust vibration intensity and patterns.
3. Medical Application: Can be programmed for specific rehabilitation exercises, providing tactile feedback for patients.

### Advantages

Enhances music appreciation for deaf and hard of hearing individuals.
Integrates seamlessly with existing Apple technology.
Potential for medical applications, particularly in rehabilitation.
Customizable vibration patterns for various uses.

1. A haptic glove system comprising:
A pair of gloves embedded with taptic engines.
A Bluetooth module for wireless connection to a smartphone.
A control unit with wiring and controllers for processing haptic signals.
A power source.

2. The system of claim 1, wherein the haptic gloves synchronize with a smartphone's haptic music feature to produce vibrations corresponding to music playback.

3. The system of claim 1, wherein the vibration patterns are customizable for different applications, including music appreciation and medical rehabilitation.

4. A method of enhancing music experience for hearing-impaired individuals using the haptic glove system of claim 1.

5. A method of providing tactile feedback for medical rehabilitation exercises using the haptic glove system of claim 1.
